(19) United States
(12) Patent Application Publication
(10) Pub. No.: US 2024/0417416 A1
(43) Pub. Date: Dec. 19, 2024

(54) CATALYTIC METHOD FOR THE PRODUCTION OF DIFRUCTOSE ANHYDRIDE

(71) Applicants: Wisconsin Alumni Research Foundation, Madison, WI (US); University of Puerto Rico, Mayagüez (PR)

(72) Inventors: Ive Hermans, Middleton, WI (US); Nelson Cardona Martínez, Mayagüez (PR); Isabel Hortal-Sánchez, Mayagüez (PR); Edgard Lebron Rodriguez, Madison, WI (US); Wei-Shang Lo, Madison, WI (US); Faysal Ibrahim, Madison, WI (US)

(21) Appl. No.: 18/745,797

(22) Filed: Jun. 17, 2024

Related U.S. Application Data
(60) Provisional application No. 63/508,698, filed on Jun. 16, 2023.

(51) Int. Cl. C07H 9/04; B01J 21/08; B01J 29/70; B01J 31/02

(52) U.S. Cl. CPC C07H 9/04; B01J 21/08; B01J 29/7007; B01J 29/7057; B01J 31/0225; B01J 2229/183

(57) ABSTRACT

Disclosed herein are methods, compositions, and reactor systems for producing difructose anhydride. The method comprises contacting a solution comprising fructose and gamma valerolactone with an acidic catalyst at an effective temperature for an effective contact time to produce difructose anhydride.

### CROSS-REFERENCE TO RELATED APPLICATIONS

The present application claims benefit of priority to U.S. Patent Application Ser. No. 63/508,698, filed Jun. 16, 2023, the contents of which is incorporated by reference in its entirety.

### STATEMENT REGARDING FEDERALLY SPONSORED RESEARCH

This invention was made with government support under DMR-1827894 awarded by the National Science Foundation. The government has certain rights in the invention.

### FIELD OF THE INVENTION

The disclosed technology is generally directed to the catalytic production of difructose anhydride.

### BACKGROUND OF THE INVENTION

Difructose anhydrides have gained attention as functional sweeteners due to their potential prebiotic effects, antioxidant and immunoregulatory properties, and their ability to enhance calcium absorption in vertebrates. DFA III or alpha-D-fructofuranose beta-D-fructofuranose 1,2′:2,3′-dianhydride is a cyclic disaccharide molecule composed of two reciprocal glycosidic linkages formed by condensation of two fructose molecules. Numerous studies indicate that DFA III displays multiple functional properties such as treatments for anemia, colon and liver cancer and osteoporosis. It also promotes the improvement of the immune system and the absorption of bioactive compounds. It is a low-calorie sweetener which may be used as a substitute of sucrose. Thus, DF As have commercial value in the food, nutraceutical, and pharmaceutical industries.

The enzymatic conversion of inulin is the main industrial route for the production of DFA III. Inulin is significantly more expensive than other carbohydrates, sucrose as a reactant also shows drawbacks such as low thermal stability, enzymes used are dependent on the substrate converted, and the process is expensive. Accordingly, there is a need in the art for improved methods for the production of DFAs.

### BRIEF SUMMARY OF THE INVENTION

Disclosed herein is a catalytic method for the production of a difructose anhydride, such as DFA III. The method comprises contacting a solution comprising fructose and gamma valerolactone with an acidic catalyst at an effective temperature for an effective contact time to produce the difructose anhydride. The acidic catalyst may be an acidic heterogenous or homogeneous catalyst, including acidic heterogenous catalyst having Lewis or Brønsted acidic sites.

Another aspect of the technology provides for composition for producing difructose anhydrides, such as DFA III. The composition comprises a solution, the solution comprising fructose and gamma valerolactone, in contact with an acidic catalyst.

Yet another aspect of the technology provides for a reactor system for producing a difructose anhydride, such as DFA III. The reactor system comprises a reactor vessel having a composition for producing a difructose anhydride therein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Effect of Brønsted and Lewis acidity on fructose conversion. Reaction conditions: 398 K, 15 min, 46 mg of fructose, 3.019 g of GVL, and 2.4 μmol of active sites.

FIG. 2. Effect of temperature on fructose conversion for Sn-Beta zeolite. Reaction conditions: 15 min, 46 mg of fructose, 3.019 g of GVL, and 2.4 μmol of Sn sites.

FIG. 3. Effect of temperature on fructose conversion for Al-Beta zeolite. Reaction conditions: 15 min, 46 mg of fructose, 3.019 g of GVL, and 2.4 μmol of Al sites.

FIG. 4. Sodium Cationization Electrospray Ionization Orbitrap Mass Spectrometry shows presence of DFA+Na⁺.

FIG. 5. ¹H NMR spectra in D₂O of (A) DFAIII standard, (B) DFA product mixture, and (C) spectra overlay.

FIG. 6. Fructose conversion with alternative Brønsted acid catalysts. Reaction conditions: 46 mg of fructose, 3.019 g of GVL, and 2.4 μmol of acidic sites.

FIG. 7. Effect of reaction time on fructose conversion for Sn-Beta zeolite. Reaction conditions: 46 mg of fructose, 3.019 g of GVL, and 2.4 μmol of Sn sites.

FIG. 8. Effect of reaction time on fructose conversion for Al-Beta zeolite. Reaction conditions: 46 mg of fructose, 3.019 g of GVL, and 2.4 μmol of Al sites.

FIG. 9. Effect of Al-Beta zeolite reuse and regeneration on fructose conversion to DFA. Reaction conditions: 1.5% wt fructose in GVL, 413 K and 2.4 μmol of acid sites.

### DETAILED DESCRIPTION OF THE INVENTION

Disclosed herein is a catalytic method for the production of DFA. The presently disclosed technology allows for the conversion of fructose to DFAs with conversion yields exceeding 70%. Advantageously the method utilizes a comparatively inexpensive starting material than inulin, which is the starting material for the main industrial route to DFA III production. Moreover, the method is not limited by the viscosity of the reactive mixture.

There are four types of enzymatically converted DFAs differentiated by the linkage between the fructose monomers: DFA I, α-D-fructofuranose-β-D-fructofuranose-2′,1:2,1′-dianhydride; III, α-D-fructofuranose-β-D-fructofuranose-2′,1:2,3′-dianhydride

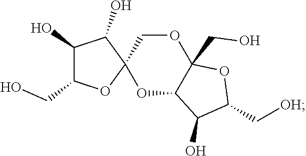 [SMILES: OC[C@H]1O[C@@]2(CO[C@]3(CO)O[C@H](CO)[C@@H](O)[C@@H]3O2)[C@@H](O)[C@@H]1O]

IV, β-D-fructofuranose-β-D-fructofuranose-2′,6:2,6′-dianhydride, and V, α-D-fructofuranose-β-D-fructofuranose-2′,6:2,1′-dianhydride. DFA I, III, and V are converted usually from inulin (β-2,1 fructan) by the exo-acting transfructosylation of inulin fructotransferase (IFTase, EC 4.2.2.18), and DFA IV is converted from levan (β-2,6 fructan) by levan fructotransferase (LFTase, EC 4.2.2.16). DFA III has been commercialized as the functional food product and has demonstrated beneficial biological activity.

Production of difructose anhydride is accomplished by contacting a fructose solution with an acidic catalyst at an effective temperature for an effective contact time to produce difructose anhydride, such as DFA III. The fructose solution comprises gamma valerolactone (GVL), the structure of which is

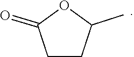

GVL can act as a solvent. The concentration of fructose in GVL in the fructose solution may be varied. Suitably, the concentration of fructose in the solution may be between 0.5 wt % and 5.0 wt %. In some embodiments, the concentration of fructose is greater 0.50 wt %, 0.60 wt %, 0.70 wt %, 0.80 wt %, 0.90 wt %, 1.00 wt %, 1.10 wt %, 1.20 wt %, 1.30 wt %, 1.40 wt %, or 1.50 wt % and less than 5.00 wt %, 4.75 wt %, 4.50 wt %, 4.25 wt %, 4.00 wt %, 3.75 wt %, 3.50 wt %, 3.25 wt %, 3.00 wt %, 2.75 wt %, 2.50 wt %, 2.25 wt %, 2.00 wt %, 1.75 wt %, or 1.50 wt %. The Examples demonstrate the use of a fructose-GVL solution having a concentration of fructose between 0.75 and 2.25 wt %.

The fructose-GVL solution may optionally comprise one or more additional components. The one or more additional components may be included in the fructose-GVL solution prior to contacting the solution with a heterogeneous catalyst or may be generated in situ as a byproduct of the reaction of fructose to GVL.

In some embodiments, the fructose-GVL solution consists essentially of fructose and GVL prior to contacting the solution with an acidic catalyst. When the fructose-GVL solution consists essentially of fructose and GVL prior to contact with the catalyst, the solution may comprise less than 2%, less than 1%, less than 0.5%, less than 0.2%, less than 0.1%, or undetectable amounts of one or more additional components.

The acidic catalyst may be a heterogeneous catalyst. The heterogeneous catalyst has a different phase than the fructose. Suitably the heterogeneous catalyst is a solid catalyst, and the fructose is in a liquid solution. The heterogeneous catalyst utilized in the disclosed methods comprises acidic sites. The acidic sites may be Brønsted acidic sites, Lewis acidic sites, or a combination thereof. Brønsted acid sites comprise polarized hydroxyl groups or H⁺ donors in the heterogenous catalyst. Lewis acid sites can accept electron pairs from guest molecules.

The heterogeneous catalyst may be any material that possesses acidic sites. Exemplary heterogenous catalysts, include without limitation, zeolites or mesoporous materials. Zeolites are traditionally referred to as a family of open-framework materials consisting of orderly distributed micropores in molecular dimensions. The frameworks of zeolites are built from the connections of corner-sharing TO₄ tetrahedra (“T” denotes tetrahedrally coordinated elements such as Si, Al, or P, etc.), and different ways of tetrahedra connection lead to a diversity of zeolite framework types based on various compositions. Various distinct zeolite framework types have been identified in natural or synthetic zeolites, each of which has been assigned a three-letter code such as BEA (e.g., beta), FAU (e.g., X, Y, faujasite), MOR (e.g., mordenite), HEU (e.g., heulandite, clinoptilolite), MFI (e.g., ZSM-5, silicalite-1), CHA (e.g., SAPO-34, SSZ, chabazite), or LTA (e.g., A). The frameworks of zeolites can be decomposed into rings of different sizes, which correspond to the pore opening, or pore windows of zeolites. According to their largest pore windows, zeolites can be categorized into small-pore (≤8-ring), medium-pore (10-ring), large-pore (12-ring), and extra-large-pore zeolites (>12-ring). The negative charges of zeolite frameworks are usually compensated by extra-framework mono- or di-valent cations, which can be exchanged by other cations. The additional species (e.g., H₂O) in the pores of zeolites can be removed, leaving void space for the selective entrance of guest species of suitable size, shape, and polarity. This is known as the molecular-sieving effect of zeolites. Moreover, the spatial confinement of zeolite pores coupled with catalytically active sites in their frameworks can endow zeolites with unique shape-selective catalysis toward the formation of specific products.

Mesoporous materials have pores with diameters between 2 and 50 nm. Mesoporous materials include porous silica, alumina, or oxide materials. Mesoporous materials have high specific surface areas and can display a wide range of morphologies, such as spheres, rods, disks, and powders. Mesoporous materials may have ordered pores but may also have disordered pores. Exemplary mesoporous silica materials include, without limitation, SBA15, SBA-16, MCM 41, and MCM 48.

The acidic character of the heterogenous catalyst may be controlled through synthetic methods during initial catalyst preparation or post-synthetic methods, such as isomorphic substitution or surface functionalization.

Atoms within a zeolite framework may be substituted for another to produce the desired amount and type or acidity. For example, Si atoms in zeolite frameworks substituted with coordinated Sn, Ti, or Zr can produce Lewis acid sites. For another example, Si atoms in zeolite frameworks substituted with coordinated Al can produce Brønsted acid sites. For yet another example, Si atoms in zeolite frameworks substituted with coordinated Sn and Al can produce Lewis and Brønsted acid sites. Examples of such substituted zeolite frameworks include the Sn-beta and Al-beta zeolites utilized in the examples.

Surface functionalization may be accomplished by post-grafting or other suitable technology. For example, propylsulfonic acid may be used to functionalize mesoporous silica SBA-15 with different amounts of the propylsulfonic acid additive to provides —PrSO₃H acid sites on the surface of the mesoporous material.

The acidic catalyst may be a homogenous catalyst. The Examples demonstrate the use of triflic or sulfuric acid as homogenous catalyst, but other acidic homogenous catalyst may also be used.

Fructose is contacted with the catalyst at an effective temperature for producing DFA. The effective temperature of contact between the fructose or heterogeneous catalyst may be selected to result in a high concentration of DFA III and limit byproduct formation. Suitably, the effective temperature may be between 90° C. (363 K) and 165° C. (438 K). In some embodiments, the effective temperature is between about 95° C. (368 K) and 160° C. (433 K), 100° C. (373 K) and 155° C. (428 K), 105° C. (378 K) and 150° C. (423 K), 110° C. (383 K) and 145° C. (418 K), or 115° C. (388 K) and 140° C. (413 K).

The methods may be practiced in a batch or continuous manner. In either batch or continuous operation, the effective time of contact between the fructose or heterogeneous catalyst may be selected to result in a high concentration of DFAs and limit byproduct formation. Suitably, the effective contact time may be less than 4 hours. In some embodiments, the contact time is between about 1 min and 3 hours, 1 min and 2 hours, 1 min and 1 hour, 1 min and 30 mins, 1 min and 25 mins, 1 min and 20 min, or about 1 min and 15 mins.

The effective temperature and effective contact time may be selected with regard with each other to maximize conversion or DFAs production. The effective temperature and effective contact time result in at least 75%, 80%, 85%, 90%, or 95% conversion of fructose. The effective temperature and effective contact time result in a product yield for DFAs of at least 30%, 35%, 40%, 45%, 50, or 55%. In some embodiments, the effective temperature and effective contact time results in at least 75%, 80%, 85%, 90%, or 95% conversion of fructose and a product yield for DFA III of at least 30%, 35%, 40%, 45%, 50, 55%, 60%, 65%, or 70%.

In other instances, the effective temperature or effective time may be independently selected without regard for the other.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention, and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Illustrative Embodiments

The following embodiments are illustrative and should not be interpreted to limit the scope of the claimed subject matter.

Embodiment 1. A method for producing a difructose anhydride comprising contacting a solution comprising fructose and gamma valerolactone with an acidic catalyst at an effective temperature for an effective contact time to produce the difructose anhydride.

Embodiment 2. The method of embodiment 1, wherein the acidic catalyst is an acidic heterogenous catalyst.

Embodiment 3. The method of embodiment 2, wherein the acidic heterogenous catalyst comprises Brønsted acidic sites.

Embodiment 4. The method of embodiment 2, wherein the acidic heterogenous catalyst comprises Lewis acidic sites.

Embodiment 5. The method of embodiment 2, wherein the acidic heterogenous catalyst comprises a zeolite.

Embodiment 6. The method of embodiment 5, wherein the zeolite is a beta zeolite.

Embodiment 7. The method of embodiment 6, wherein the zeolite comprises Brønsted acidic sites.

Embodiment 8. The method of embodiment 7, wherein the zeolite comprises an Al-beta zeolite.

Embodiment 9. The method of embodiment 6, wherein the zeolite comprises Lewis acidic sites.

Embodiment 10. The method of embodiment 9, wherein the zeolite comprises a Sn-beta zeolite.

Embodiment 11. The method of embodiment 2, wherein the acidic heterogenous catalyst is a mesoporous material.

Embodiment 12. The method of embodiment 11, wherein the mesoporous material is a mesoporous silica.

Embodiment 13. The method of embodiment 11, wherein the acidic heterogeneous catalyst comprises Brønsted acidic sites.

Embodiment 14. The method of embodiment 11, wherein the acidic heterogenous catalyst comprises Lewis acidic sites.

Embodiment 15. The method of embodiment 11, wherein the mesoporous material comprises SBA-15.

Embodiment 16. The method of embodiment 15, wherein the SBA-15 is functionalized with propylsulfonic acid.

Embodiment 17. The method of embodiment 1, wherein the acidic catalyst is a homogenous acidic catalyst.

Embodiment 18. The method of any one of embodiments 1-17, wherein the concentration of fructose in GVL is between 0.0.5 wt % and 0.5.0 wt %.

Embodiment 19. The method of embodiment 18, wherein the weight ratio of fructose to gamma valerolactone in the solution is between 0.75 wt % and 2.25 wt %.

Embodiment 20. The method of any one of embodiments 1-19, wherein the effective contact time is less than 4 hours.

Embodiment 21. The method of embodiment 20, wherein the effective contact time is between 1 minute and 30 minutes.

Embodiment 22. The method of any one of embodiments 1-21, wherein the effective temperature is a temperature between 90° C. and 165° C.

Embodiment 23. A composition for producing a difructose anhydride comprising a solution, the solution comprising fructose and gamma valerolactone, in contact with the acidic catalyst according to any one of embodiments 1-19.

Embodiment 24. A reactor system for producing a difructose anhydride comprising a reaction vessel having the composition according to embodiment 23 therein.

Embodiment 25. The reactor system of embodiment 24 in thermal communication with a heater operably configured to heat the composition to an effective temperature to produce the difructose anhydride.

Embodiment 26. The reactor system of any one of embodiments 24-25, the reactor system operably configured to allow the solution and the acidic catalyst to contact each other for an effective contact time to produce the difructose anhydride.

### EXAMPLES

The Examples demonstrate the successful conversion of fructose to DFA with a variety of acidic homogeneous and heterogenous catalysts having both Brønsted and Lewis acidic sites. Beta zeolites incorporating Sn and Al demonstrated the highest activity, but moderate activity was also shown for a silica-based mesoporous material.

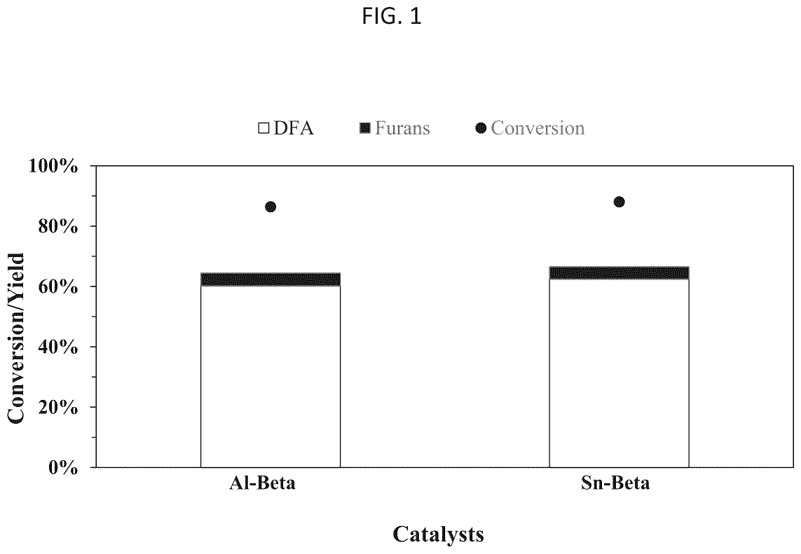

The Examples demonstrate the production of DFAs with yields exceeding 74% using acidic heterogenous catalysts, such as Brønsted or Lewis acidic Beta zeolites, in pure gamma valerolactone (GVL) as solvent. After 15 minutes, conversion around 87% for both Sn-Beta and Al-Beta zeolites was observed (FIG. 1). For Sn-Beta (Lewis acid zeolite) the yield towards DFA was 62% and the yield towards furans which includes 5-hydroxymethyl furfural (HMF) and 2-furaldehyde was 4%. For Al-Beta (Brønsted acid zeolite) the yield towards DFA was 60% and the yield towards furans was 4%. Catalysts with both types of acidity are able to catalyze the reaction with a reduction in the production of furans when Lewis acidity is used instead of Brønsted acidity at temperatures higher than 398 K. Brønsted acidity generally promotes the dehydration of carbohydrates to furans at higher temperatures.

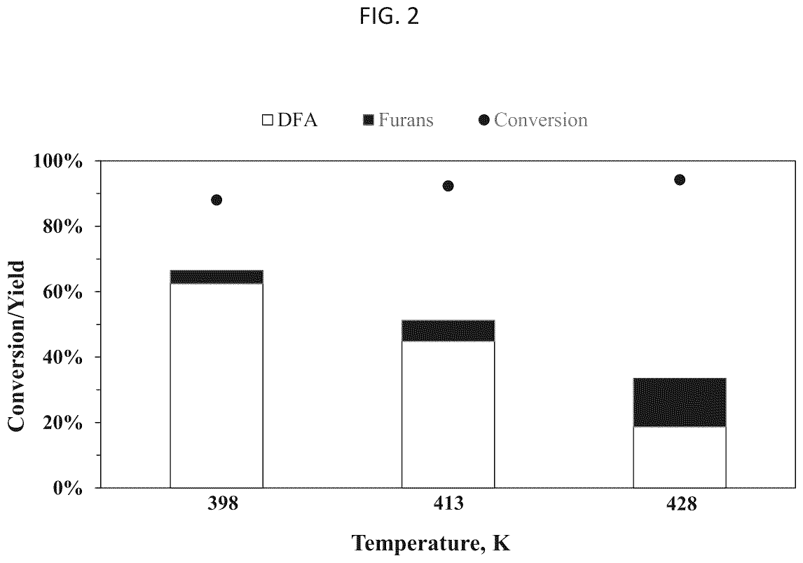

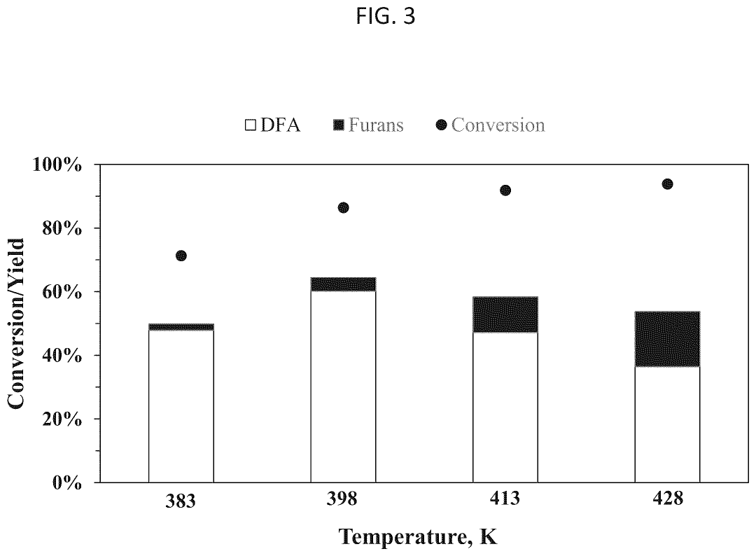

The reaction temperature had an effect on fructose conversion and on the products distribution (FIGS. 2 and 3). For both Brønsted and Lewis acid Beta zeolite catalysts an increase in fructose conversion with an increase in the reaction temperature was observed. For Al-Beta the yield towards DFA passed through a maximum (60%) around 398 K. For Sn-Beta zeolite the yield towards DFA was 62% at 398 K. For both catalysts the yield towards furans increased with an increase in reaction temperature. For Sn-Beta increasing the temperature to 428 K caused a reduction in the yield towards DFAs from 45% to 19% (FIG. 2) and from 47% to 36% for Al-Beta (FIG. 3), with an increase in furans yield from 6% to 15% for Sn-Beta and for Al-Beta from 11% to 17%. Decreasing the temperature to 398 K also caused a higher production in DFAs yields. For Sn-Beta the increase was from 45% to 62% and for Al-Beta from 47% to 60%. Also, a decrease in the furans yield from 6% to 4% for Sn-Beta and from 11% to 4% for Al-Beta was observed.

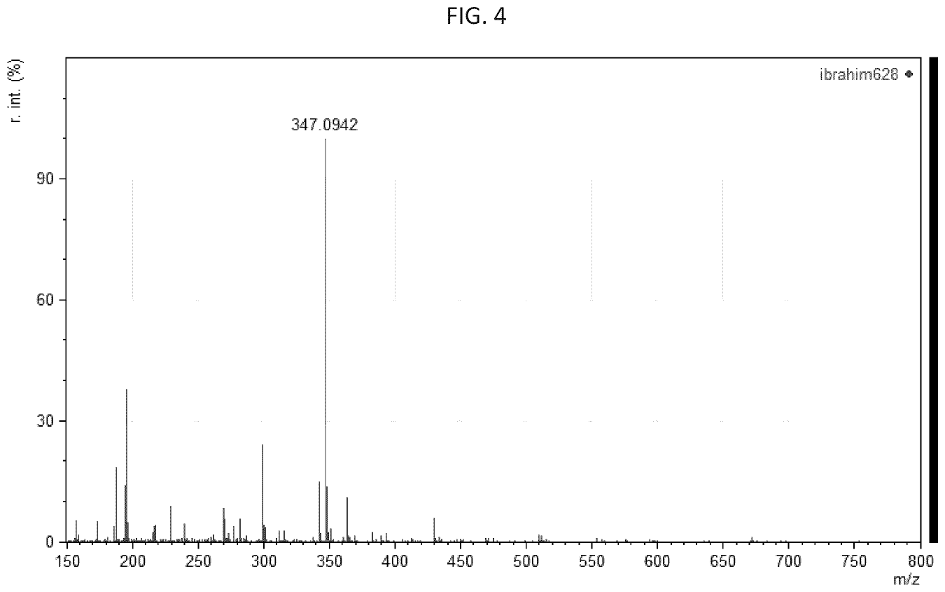

FIG. 4 shows a mass spectrum obtained using Sodium Cationization Electrospray Ionization Orbitrap Mass Spectrometry. This MS method creates a sodium adduct with the compound, so DFA is observed as DFA molar mass+Na molar mass, or 324.11+22.99=347.1 m/z. No prominent signals beyond the DFA m/z value are observed, demonstrating that no oligomeric or polymeric species are present.

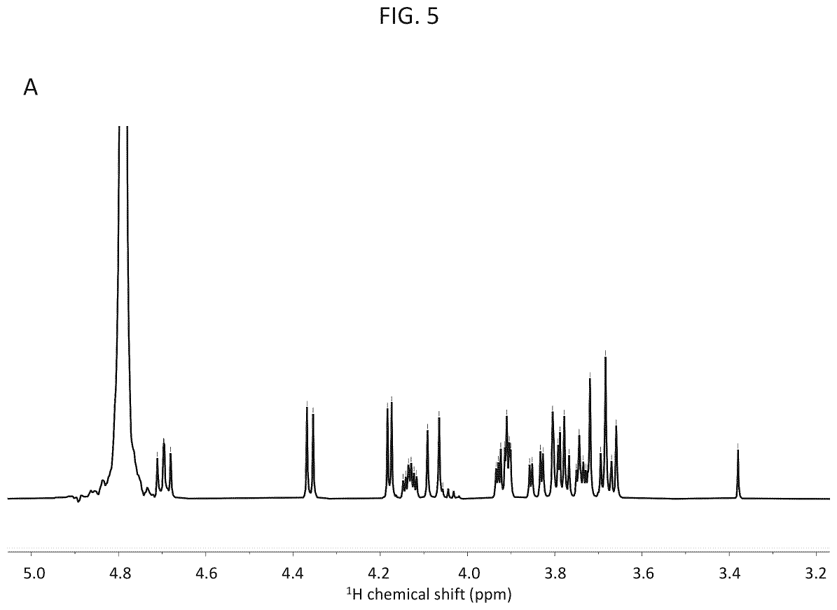

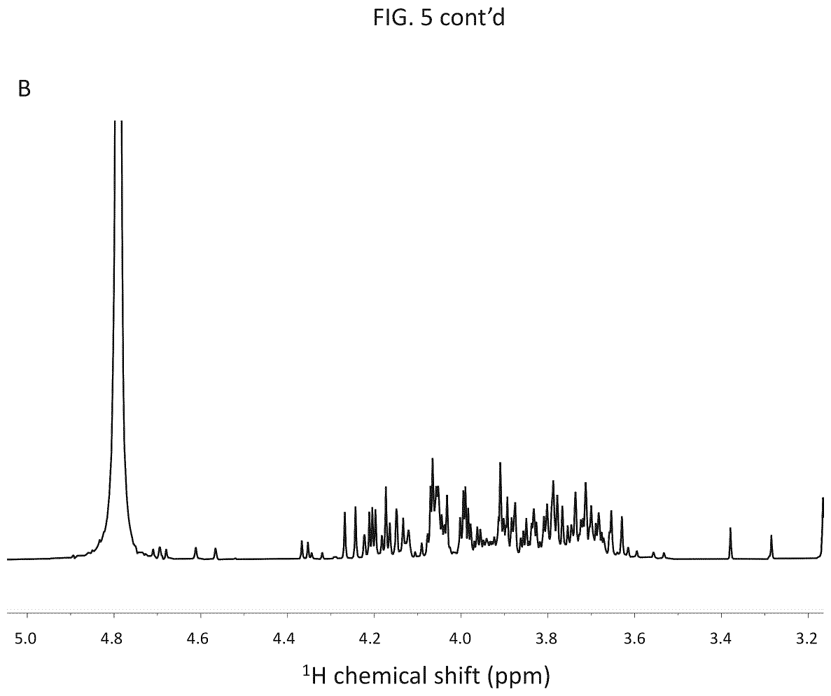

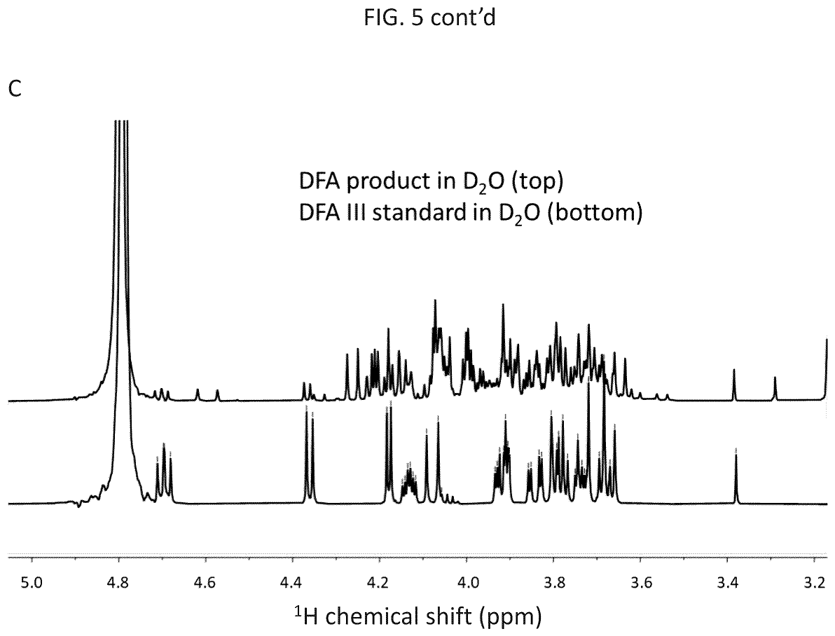

FIG. 5 shows 1H NMR spectra in D₂O demonstrating the presence of DFA, including DFA I, DFA III, and DFA IV, in the product by way of comparison with a DFA III standard (FUJIFILM Wako Pure Chemical Corporation, +97%) spectrum (FIG. 5A) and with a product spectrum (FIG. 5B). An overlay of the two spectra is shown in FIG. 5C. The sample of DFA product (Table 3, entry 6) was obtained from the reaction solution using an HPLC Waters 2695 accumulating the fractions with the adequate retention time. The sulfuric acid was removed from the HPLC solution using AMBERLITE PWA15. The solution was concentrated using lyophilization. The DFA solution was obtained from a run using 2.25 wt % fructose, 3.065 g of GVL, 398 K and 2.2 mg of Al-Beta.

No evidence of reaction after 24 h at room temperature for the catalysts discussed above using fructose as reactant and GVL as solvent was observed. At 15 minutes, and at 140° C. (413 K) without catalyst only 1% of conversion and trace amounts of DFA was observed.

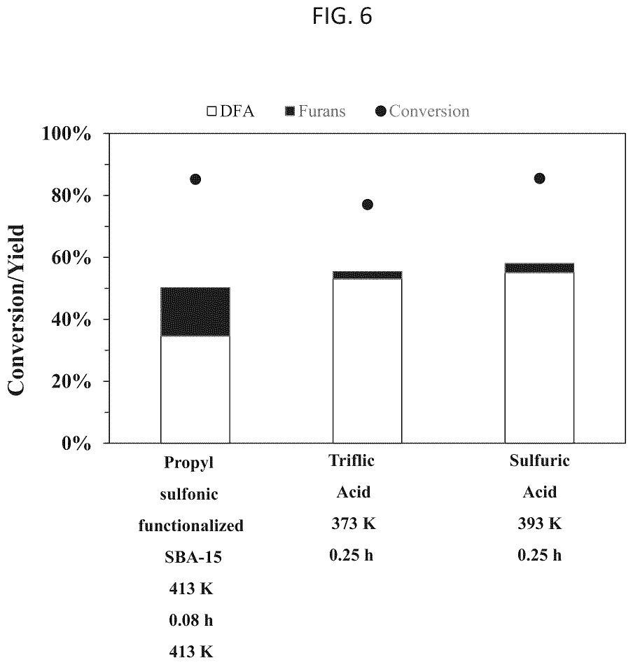

FIG. 6 demonstrates the activity of other heterogeneous and homogeneous Brønsted acidic catalysts such as propylsulfonic acid functionalized SBA-15, triflic acid and sulfuric acid They display moderate activity, demonstrating the importance of strong acid sites for fructose conversion. Weak homogeneous catalysts such as formic and acetic acid were also tested with little activity compared to strong homogeneous acids and heterogeneous acidic catalysts.

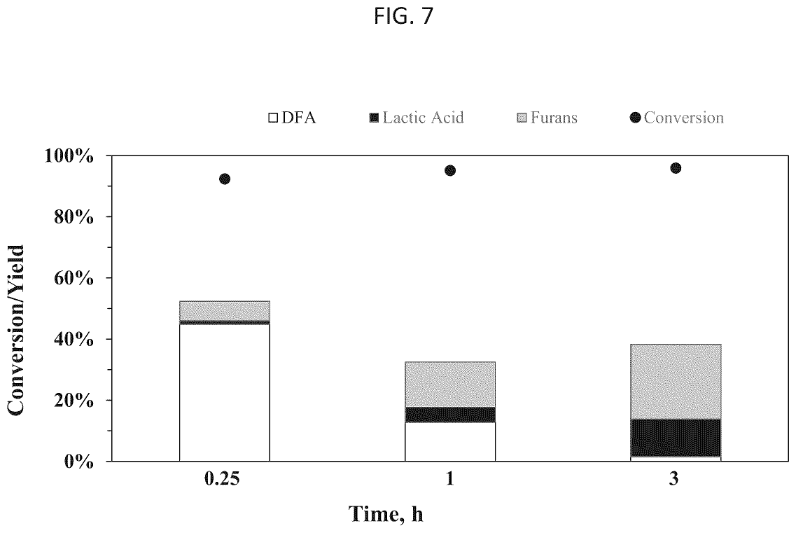

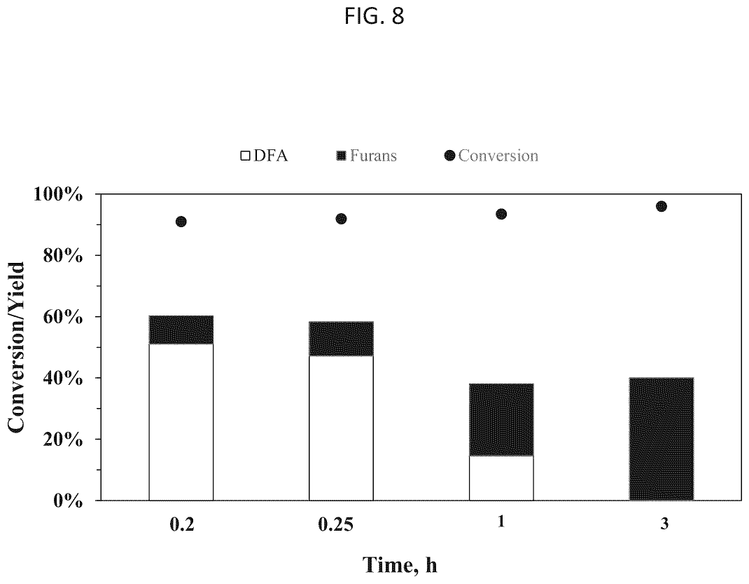

FIGS. 7 and 8 illustrate the effect of reaction time on fructose conversion to DFA. DFA production was highest at short times, e.g. 12 min or 15 min, and decreased with increasing reaction time. Although fructose conversion increases with time, such an increase is also accompanied with increasing by-product production.

### Results and Discussion

### Catalysts for DFA Production

The effectiveness of various homogeneous and heterogeneous Brønsted and Lewis acid catalysts for DFA production from fructose (Table 1) was tested. The presence of water inhibits the production of DFA. For that reason, pure polar aprotic solvents such as GVL were used.

The use of the heterogeneous Brønsted and Lewis acidic Beta zeolites (Al-Beta and Sn-Beta, respectively) as catalysts for the conversion of fructose in GVL at 413 K resulted in high DFA yields (47% and 45%) at high conversions (92%) (Table 1, entry 1 and 2). These results contrast with other reported for which selectivities higher than 45% were only found at low conversions or using the toxic, highly corrosive and environmentally unfriendly hydrofluoric acid.^{1,2,3} For Sn-Beta lower furans yield than for Al-Beta was observed because the Sn-Beta sample has a lower concentration of Brønsted acid sites that promote the dehydration of carbohydrates to furans, which include HMF and 2-furaldehyde.⁴

To elucidate how acidity affects the reaction performance, dealuminated zeolite samples were also studied. The synthesis method that was used to prepare Sn-Beta zeolite involved a dealumination step that removed most of the aluminum. A first run utilizing the same mass and conditions used during the Al-Beta run discussed previously gave a 15% conversion with 9% DFA yield (Table 2, entry 1). During the dealumination, the aluminum content decreased from 2.97% to 0.06%, but the acidity measured by ammonia TPD was reduced in a lower proportion, only 6 times. To obtain the same acidity as in the original Al-Beta sample the amount of catalyst was increased to 0.014 g. Under those conditions, the conversion increased to 85% and the yield towards DFA to 54% (Table 2, entry 2), which is a higher value than the one observed for the original Al-Beta sample. Thus, increasing by only six times the amount of catalyst, a high conversion was observed, lower furans yield but higher yield towards DFA using a dealuminated zeolite rather than the parent Al-Beta. The higher DFA yield obtained for the dealuminated material may be explained by considering that the lower amount of acid sites favored the DFA accumulation since the intermediate step from DFA to HMF results inhibited, supposing that DFA acts as an intermediate in HMF production.^{1,2} To test the effect of the degree of dealumination a second dealuminated Beta zeolite sample that was dealuminated twice under more severe conditions was also studied. For that sample, the activity was lower than the sample dealuminated once and adding the same acidity used in the other runs only gave 29% of conversion with 10% in DFA yield (Table 2, entry 4). Those results suggest that the more severe dealumination eliminates acid sites that are important for the formation of DFA.

TABLE 1
Effect of catalyst acid strength on fructose conversion to DFA.

| | | | Tem- | | Product | |
|---|---|---|---|---|---|---|
| | | Time | perature | | Yield | |
| Entry | Catalyst | (h) | (K) | Conversion | DFA | Furans |
| 1 | Al-Beta | 0.25 | 413 | 92% | 47% | 11% |
| 2 | Sn-Beta | 0.25 | 413 | 92% | 45% | 6% |
| 3 | Triflic Acid | 0.25 | 373 | 77% | 53% | 2% |
| 4 | | 0.25 | 413 | 100% | 0% | 61% |
| 5 | Sulfuric Acid | 0.25 | 393 | 85% | 55% | 3% |
| 6 | | 0.25 | 413 | 96% | 5% | 43% |
| 7 | Formic Acid | 0.25 | 413 | 9% | 1% | 0% |
| 8 | | 3.00 | 413 | 65% | 33% | 1% |
| 9 ^{a} | | 0.25 | 413 | 25% | 6% | 0% |
| 10 ^{a} | | 3.00 | 413 | 79% | 42% | 4% |
| 11 | Acetic Acid | 0.25 | 413 | 5% | 0% | 0% |

Reaction conditions: 1.5% wt fructose in GVL and 2.4 μmol of acid sites.
^{a} A concentration of 0.08M of formic acid was used as a catalyst.

TABLE 2
Other materials used for fructose conversion to DFA.

| | | Amount | | | |
|---|---|---|---|---|---|
| | | of | | | |
| | | catalyst | Conversion | Product Yield (%) | |
| Entry | Catalyst | (mg) | (%) | DFA | Furans |
| 1 | DeAl-Beta | 2.2 | 15% | 9% | 1% |
| 2 | | 14 | 85% | 54% | 4% |
| 3 | DeAl-Betax2 | 2.2 | 4% | 2% | 0% |
| 4 | | 18 | 29% | 10% | 0% |
| 5 | Si-Beta | 2.2 | 1% | 1% | 0% |
| 6 | SiO₂ | 9.8 | 2% | 0.49% | 0% |
| 7 | SBA-15 | 2.2 | 1% | 0.30% | 0% |
| 8^{a} | SnCl₄•5H₂O | 0.8 | 96% | 3% | 33% |
| 9 | Tin acetate | 0.6 | 76% | 47% | 3% |
| | (II) | | | | |
| 10^{a} | Propyl | 0.4 | 85% | 35% | 16% |
| 11 | sulfonic | | 94% | 12% | 19% |
| | functionalized | | | | |
| | SBA-15 | | | | |

Reaction conditions: 413 K. in GVL, 1.5% wt fructose and 15 min of reaction.
^{a}Time of reaction: 5 min (probably there was not sufficient time to reach the desired reaction temperature).

Heterogenous catalysts are usually preferred over homogeneous catalysts because they are more easily separated from the product solution by filtration leading to a more sustainable and economically viable process. To obtain a better understanding of the conversion of fructose to DFA, homogeneous catalysts were also studied. As homogeneous catalysts, some Brønsted acids were selected with pKa values in water spanning from strong to weak acids including triflic acid (−14), sulfuric acid (−2.8), formic acid (3.75) and acetic acid (4.75).⁵ The stronger the catalyst acidity the higher the catalytic activity observed under the experimental conditions described herein. Runs with strong Brønsted acid catalysts at 413 K (Table 1, entries 4 and 6) displayed almost complete conversion, high furans yield (>40%) and low DFA yields. A decrease in the reaction temperature (Table 1, entries 3 and 5) improved the yield towards DFA and decreased the furans yield. For instance, from no DFA detection to 53% yield at 77% conversion for the run catalyzed by triflic acid at 373 K.² Weak Brønsted acids at 413 K require more time than 15 minutes to produce significant DFA yields (Table 1, entry 7-11).

The results discussed above suggest that both heterogeneous Brønsted and Lewis acid catalysts are effective for the production of DFA at mild conditions and provide additional evidence that DFA acts as an intermediate in HMF production.

### Effect of Temperature

The reaction temperature had an effect on fructose conversion and the product distribution (FIGS. 2 and 3). For both Brønsted and Lewis acid Beta zeolite catalysts an increase in fructose conversion with an increase in the reaction temperature was observed. For Al-Beta the yield towards DFA passed by a maximum (60%) around 398 K. For Sn-Beta zeolite the yield towards DFA was 62% at 398 K. For both catalysts the yield towards furans increased with an increase in reaction temperature.

### Effect of Catalyst and Fructose Loadings

When the loading was increased from 0.5 mg to 2.2 mg for Al-Beta zeolite, the conversion also slightly increased from 89 to 92% (Table 3, entries 1-3). The small effect on conversion suggested that there were sufficient catalytic sites available for the reactant in the system even when a low catalyst amount was used. However, the availability of more active sites displayed detrimental effects on DFA production since it shifted the reaction towards higher furan yields which is in concordance with the above discussion about the dealuminated and the original Beta zeolite.

For economic viability, it is desirable to operate at a high substrate: catalyst mass ratio. For Al-Beta zeolite at 398 K and 0.75 wt % fructose, the conversion was 77% with 50% DFA yield (Table 3, entry 4). For 1.5 and 2.25 wt % fructose, the conversion and the DFA yield increased compared to 0.75 wt % and were about 87% and 60%, respectively for both fructose loadings (Table 3, entries 5 and 6). The fructose: catalyst mass ratio used in this study was high compared with other similar studies, in which ratios of 4:1 to 1:2 were usual.^{2, 6, 7}

The method achieved DFA yields as high as 74% using a mild reaction temperature (398 K) and with a mass ratio of fructose:Al-Beta catalyst of 92:1. These results were achieved under the following conditions: 1.5% wt fructose, 50 mL of GVL, 38.3 mg of Al-Beta and 125° C. (398 K) (equivalent to table 3, entry 5). The reaction was conducted at atmospheric pressure using a three neck round flask under a constant stream of helium continuously bubbling through the system to attempt the removal of the maximum amount of water possible and carry it away instead of using autogenous pressure as in Table 3, entry 5. The conversion was 95% with 74% yield of DFA and 5% yield of furans.

The effect of reaction time was also assessed by separate batch experiments for Al-Beta and Sn-Beta catalysts (Table 3, entries 7-12). For Al-Beta zeolite the fructose conversion increased from 91% to 96% as the reaction time was increased from 12 min to 3 h at 413 K (Table 3, entries 7-9). The DFA and glucose yields decreased with reaction time, with a DFA maximum of 51% after 12 min of reaction. Increasing the reaction time to 1 h reduced the DFA yield to 15% and after 3 h of reaction, DFA was not detected. On the other hand, the amount of levoglucosan (LGA or AGP) increased with reaction time. AGP is produced from glucose dehydration. The furans yield increased from 9% to 40%. For Sn-Beta zeolite similar tendencies were observed. The main difference was that because of its Lewis acidity, for reaction times higher than 15 min significant amounts of lactic acid were observed and the furans yields were lower than for Al-Beta zeolite at similar conversions. For instance, after 3 h of reaction, Sn-Beta displayed a lactic acid yield of 12% with a furans yield of 24% (Table 3, entry 12).

TABLE 3
Effect of reaction conditions on DFA production.

| | | Fructose | Catalyst | Tem- | Conv- | Yield | |
|---|---|---|---|---|---|---|---|
| | Time | loading | loading | perature | ersion | (%) | |
| Entry | (h) | (%) | (mg) | (K) | (%) | DFA | Furans |
| 1 | 0.25 | 1.5 | 0.5 | 413 | 89% | 62% | 4% |
| 2 | | | 1.1 | | 91% | 57% | 7% |
| 3 | | | 2.2 | | 92% | 47% | 11% |
| 4 | 0.25 | 0.75 | 2.2 | 398 | 77% | 50% | 6% |
| 5 | | 1.5 | | | 86% | 60% | 4% |
| 6 | | 2.25 | | | 88% | 60% | 3% |
| 7 | 0.2 | 1.5 | 2.2 | 413 | 91% | 51% | 9% |
| 8 | 1 | | | | 93% | 15% | 23% |
| 9 | 3 | | | | 96% | 0% | 40% |
| 10 ^{a} | 0.25 | 1.5 | 10.1 | 413 | 92% | 45% | 6% |
| 11 ^{a} | 1 | | | | 95% | 13% | 15% |
| 12 ^{a} | 3 | | | | 96% | 1% | 24% |

Reaction conditions: 1.5% wt fructose in GVL using Al-Beta as a catalyst.
^{a} _Sn-Beta was used as a catalyst.

### Effect of the Solvent

Solvents affect the reactivity, product distribution, stability of the products, lifetime of the catalyst as well as the economics of downstream separations.⁸ Unfortunately, water which is the most frequently used solvent because of its low cost, exhibited low catalytic activity and a complete inhibition in DFA production from fructose using Al-Beta or Sn-Beta as catalysts. Instead, the production of furans in the presence of Brønsted acid catalysts was observed (Table 4, entry 2) and the production of lactic acid in the presence of Lewis acid catalysts.^{7,9} For example, for Al-Beta at 413 K and 15 min of reaction in water conversion was not observed, and after 3 h of reaction only 6% conversion was observed. As mentioned before, using pure GVL augmented the catalytic activity and the DFA yield. In contrast, the presence of water even in low quantities negatively affected the production of DFA. For example, for Sn-Beta adding 2 wt % of water to pure GVL caused a reduction of 20% in fructose conversion and 42% in DFA yield (Table 4, entry 5). Under those conditions, a yield of lactic acid of 4% was observed.

Regarding GVL stability, Horvath's group studied the solvent under neutral, acidic, and basic conditions and although dry GVL is stable for several weeks at 423 K, its decomposition is possible with the suitable catalyst and can decompose to 4-hydroxyvaleric acid (4-HVA), which has a pKa of 4.59, similar to the pKa of acetic acid and because it is used as a solvent the concentration of the decomposition products may be higher than the concentration of the reagent or the catalyst precursor and can influence the reaction results.¹⁰ Besides, levulinic acid was also detected as an impurity and its concentration increased after the reaction with acid catalysts, hindering its quantification since levulinic acid can also be produced as a degradation product from HMF.¹¹ The stability of GVL at 413 K was tested. When there was no catalyst after 3 h of reaction the conversion was lower than when Al-Beta or Sn-Beta zeolites was used as a catalyst, and not only detected 4-HVA or levulinic but also observed trace amounts of numerous unidentified species.

Since GVL slowly degrades under the reaction conditions described herein, ethylene carbonate (EC) and acetone were also studied which are also polar aprotic solvents. EC is a green solvent with low toxicity and is less expensive than GVL. Using Lewis or Brønsted acid catalysts the stability of EC at 413 K was tested. It was more stable than GVL after 3 h of reaction. Using Al-Beta in EC (Table 4, entry 6), after 15 min the conversion was 84% with a yield towards DFA of 49%. After 30 min the conversion increased to 90% and the yield to 51% (Table 4, entry 7). For Sn-Beta zeolite the conversion and yield were lower than for Al-Beta after 15 min of reaction but after 1 h of reaction the conversion was 89% and the yield increased to 64% (Table 4, entries 8 and 9). EC required at least double the reaction time needed for GVL to obtain similar results.

Acetone is inexpensive, has a low boiling point facilitating cosolvent separation, and it is considered a sustainable solvent from the point of view of safety, health and effects on the environment.^{12, 13} Unfortunately, acetone at 413 K and in the presence of an acid catalyst displayed significant yields to unidentified compounds (Table 4, entries 10 and 11). For example, for Al-Beta in acetone the conversion after 15 min of reaction was 78%, but the yield towards DFA was only 4% (Table 4, entry 9). DFA was not the main product but LGA and retro-aldol products instead (17% and 16%, respectively), which are intermediate products for the production of lactic acid. These results suggest that properties of acetone promote a different reaction pathway.

TABLE 4
Effect of solvent on fructose conversion.

| | | | Time | Conversion | Yield (%) | |
|---|---|---|---|---|---|---|
| Entry | Solvent | Catalyst | (h) | (%) | DFA | Furans |
| 1 | H₂O | Al-Beta | 0.25 | 0% | 0% | 0% |
| 2 | | | 3 | 6% | 0% | 3% |
| 3 | GVL:H₂O | Al-Beta | 0.25 | 8% | 2% | 0% |
| 4 | 9:1 | | 3 | 31% | 6% | 4% |
| 5 | GVL:H₂O | Sn-Beta | 0.25 | 72% | 29% | 4% |
| | 98:2 | | | | | |
| 6 | EC | Al-Beta | 0.25 | 84% | 49% | 3% |
| 7 | | | 0.5 | 90% | 51% | 3% |
| 8 | | Sn-Beta | 0.25 | 77% | 40% | 2% |
| 9 | | | 1 | 89% | 64% | 4% |
| 10 | Acetone | Al-Beta | 0.25 | 78% | 4% | 8% |
| 11 | | Sn-Beta | 0.25 | 59% | 18% | 1% |

Reaction conditions: 1.5% wt fructose, 413K and 2.4 μmol of acid sites.

### Reusability Study

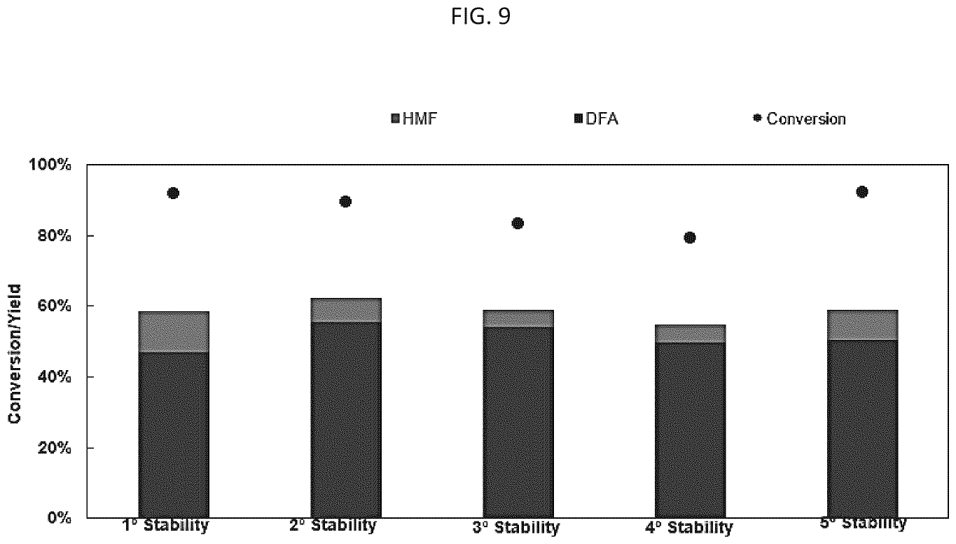

To test the stability of Al-Beta zeolite the catalyst was reused for four cycles (FIG. 9). Each run was conducted for 15 min at 413 K with 1.5 wt % fructose in GVL and 2.4 μmol of acid sites. After the reaction time the solution was quenched down in an ice-water bath, filtered the catalyst, washed it with abundant DI water and acetone and finally dried it at 393 K before reusing it. The conversion decreased after each run from 92% to 79% after the fourth cycle. However, after the second cycle, the DFA yield increased from 47% to 56% and the furans yield decreased from 11% to 6%. This result suggests again that the reduction in active sites availability inhibited the HMF production and thus favored the DFA accumulation. After the fifth cycle, the material was regenerated by a calcination for removing the deposited humins and other organic residues on the surface of the catalyst. The regenerated Al-Beta catalyst displayed a conversion of 92%, a DFA yield of 50% and furans yield of 8% which are intermediate results compared to those between the first and the second cycle, suggesting that the regeneration process could recover a part of the catalytic activity of the material.

### Catalysts for DFA Production

Non acidic crystalline or non-crystalline materials (Table 2, entry 5-7) did not display activity for the reaction from fructose to DFA, indicating the necessity of the acidity to its production.

Tin (IV) chloride pentahydrate and tin acetate (II) which are two common precursors used during the Sn-Beta synthesis were also tested as catalysts (Table 2, entry 8-9) The former has water in its structure and together with its Lewis acidity caused the production of lactic acid. A yield towards lactic acid of 3% was observed at 96% conversion. The yield towards furans was 33%. The high furans yield may be is caused because this precursor hydrolyses at moderate temperature resulting in the production of Brønsted acidic protons and thus promoting furans formation. Only 3% DFA yield was observed probably as consequence of water presence. Tin (II) acetate is used in esterification and transesterification reactions.¹⁴ It decomposes into tin oxide upon heating in the presence of water. For tin acetate a 47% DFA yield at 76% of conversion was observed.

For SBA-15 functionalized with propylsulfonic acid and thus with Brønsted acidity, after 5 min of reaction the catalyst turned black probably indicating the adsorption of carbonaceous species on the surface (Table 2, entry 10 and 11). After 15 minutes, the conversion increased from 85% to 94% compared to 5 min of reaction, and there was a reduction in the DFA yield from 35% to 12% with a slight increase in furans yield.

### Materials

The following reagents and materials were used as provided: NH₄-Beta zeolite (Alfa-Aesar, SiO₂/Al₂O₃=25), deionized water (Thermo Scientific MicroPure UV), nitric acid (Fisher-Chemical, 69.3%), dimethyl sulfoxide (Alfa Aesar, HPLC grade), tin (IV) chloride pentahydrate (Acros Organic, 98+%), 2-propanol (Fisher Chemical, A416-4), N₂ (Praxair, industrial grade), air (Praxair, zero grade), D-fructose (Acros Organics, 99%), D-glucose (Fisher Chemical, D16-500), DL-lactic acid (Acros Organic, 85%), 5-hydroxymethyl-furfural (Acros Organic, 98%), 2-furaldehyde (Across Organic, 99%), formic Acid (Acros Organic, 99%), levulinic Acid (Acros Organic, 98%), acetic acid (Fisher Chemical A38-500, glacial), difructose anhydride (III) (FUJIFILM Wako Pure Chemical Corporation, +97%), levoglucosan (Carbosynth), sulfuric acid (Fisher Chemical, 95.0-98.0%), Triflic acid (TCI, +98%), gamma-valerolactone (Sigma Aldrich, +99%), ethylene carbonate (Sigma Aldrich, 98%) and acetone (Sigma Aldrich, HPLC grade).

### Methods

### Synthesis of Sn-Beta

The grafting post-synthesis method was used to prepare the Sn-Beta catalyst. The ammonium form of the zeolite (NH₄-Beta) was first calcined under flowing dry air (zero air grade passed through Drierite® with indicator, 8 mesh) at 150 mL/min with a heating rate of 1 K/min up to 853 K for 10 h using a tube furnace to obtain the protonated form of the zeolite (Al-Beta). Before starting the dealumination, the material was dried at 393 K for 2 h under vacuum. Afterward, the zeolite sample was suspended in a 14 M aqueous solution of HNO₃ (Fisher-Chemical, 69.3%) in a round bottom flask at 353 K and stirred at 800 rpm overnight. The material was washed with abundant deionized water through a glass fiber prefilter over a Büchner funnel, connected by a rubber stopper to a Kitasato flask until the pH was neutral.

Before grafting the metal, the dealuminated sample was dried under vacuum at 393 K for 2 h and then the surface of the catalysts was activated at 423 K for 2 h to remove adsorbed water. The zeolite was added to a solution of SnCl₄·5H₂O in DMSO, which was placed in a round bottom flask at the reflux temperature under an N₂ atmosphere (industrial grade passed through Drierite® with indicator, 8 mesh) for 7 h. Then, the material was filtered, rinsed with isopropanol and dried under vacuum at 393 K. Finally, the catalyst was calcined under flowing dry air with the following heating program: 298 K to 333 K in 10 min, 333 K for 4 h, 333 K to 473 K in 50 min, 473 K for 6 h, 473 K to 823 K in 2 h, and 823 K for 6 h.

### Catalyst Characterization

The elemental composition of the samples (commercial ammonia Beta zeolite, DeAl-Beta, and Sn-Beta) were analyzed at Galbraith Laboratories (Knoxville, TN). XRD was used to obtain the crystal structure of the samples using a Bruker D8 Advance Diffractometer with Cu-Kα radiation (λ=1.5406 Å, 40 kV, 40 mA). The diffraction patterns were collected from 5° to 80° with a step size of 0.02° and a scanning rate of 1.2° per min. The UV-Vis diffuse-reflectance spectroscopy spectra were recorded using a Shimadzu UV-2600 UV-VIS spectrophotometer equipped with an MPC-2600A Multipurpose Sample Compartment. Before taking the spectra, the samples were dried at 393 K overnight under vacuum. The spectra were collected from 200 nm to 600 nm with a sampling interval of 1 nm and a fast scan speed. Barium sulfate was used as the blank reference. IR spectra were obtained on a self-supporting wafer by using a Bruker Alpha spectrometer in absorbance mode. The spectrometer was placed inside a glovebox (<1 ppm of H₂O and O₂). NH₃ Temperature Programmed Desorption (TPD) was used to determine the total acidity (μmol/g) of the samples. The methodology used was described by Harris et al. for NH₃ titration experiments. (Harris, J W. “Titration and Quantification of Open and Closed Lewis Acid Sites in Sn-Beta Zeolites That Catalyze Glucose Isomerization” J. Catal. 2016, 335, 141-154.) To study the textural properties of the zeolite samples a Micromeritics VacPrep™ 061 unit at 77K was used to obtain the nitrogen adsorption/desorption isotherms. Before these measurements, the samples were degassed at 623 K for 8 h.

### Catalytic Tests

To study the kinetics of fructose conversion to DFA 10 mL thick-walled glass batch reactors equipped with magnetic stirrers were used and heated in a temperature-controlled oil bath. For a typical run the appropriate amounts of solvent, fructose, and catalyst were placed in four reactors and placed them in an oil bath at the reaction temperature with vigorous stirring at 550 rpm and autogenous pressure or at atmospheric pressure while bubbling helium through the reaction solution. In some instances, fructose finished dissolving in situ because it has limited solubility at room temperature in the pure polar aprotic solvents. After the allotted reaction time the reaction was stopped by placing the reactors in an ice/water bath for 3 min. Then the reaction solution was diluted with water in a ratio of 1:2. To separate the catalyst from the reaction medium, the reactors were centrifuged for 15 min at 2000 rpm. Using a syringe with a 0.2 μm PTFE membrane filter aliquots of the product solution and placed them in HPLC vials. For the quantification of the products, a Waters 2695 HPLC equipped with an Aminex HPX-87H column coupled with RI-2410 and UV-486 detectors in series was used.

To calculate the conversion of fructose and the yields of products the following equations were used:

$$Conversion ( mol \% ) = \frac{moles of {fructose}_{inlet} - moles of {fructose}_{outlet}}{moles of {fructose}_{inlet}} \times 100 \qquad ( 1 )$$

$$Product Carbon Yield = \frac{moles of carbon in {product}_{i}}{moles of carbon {fructose}_{inlet}} \qquad ( 2 )$$

### Catalyst Stability

To test the stability of the Al-Beta catalyst a sample was used in four consecutive reaction cycles. After each run, the catalyst was filtered and washed with water and acetone and dried at 393 K under vacuum. After the fourth cycle, the sample was calcined using a tube furnace at 823 K for 2 h to regenerate the catalyst and remove adsorbed species. Then, the material was reused in a new run at the same reaction conditions.

### REFERENCES

(1) Cheng, T. Y.; Chao, P. Y.; Huang, Y. H.; Li, C. C.; Hsu, H. Y.; Chao, Y. S.; Tsai, T. C. Catalysis of Ordered Nanoporous Materials for Fructose Dehydration through Difructose Anhydride Intermediate. Microporous Mesoporous Mater. 2016, 233, 148-153.
(2) Huang, Y.; Chao, P. Y.; Cheng, T. Y.; Ho, Y.; Lin, C. T.; Hsu, H. Y.; Wong, J. J.; Tsai, T. C. Design of Sulfonated Mesoporous Silica Catalyst for Fructose Dehydration Guided by Difructose Anhydride Intermediate Incorporated Reaction Network. Chem. Eng. J. 2016, 283, 778-788.
(3) Defaye, J.; Gadelle, A. The Behaviour of D-Fructose and Inulin towards Anhydrous Hydrogen Fluoride. Carbohydr. Res. 1985, 136, 53-65.
(4) Mellmer, M. A.; Sener, C.; Gallo, J. M. R.; Luterbacher, J. S.; Alonso, D. M.; Dumesic, J. A. Solvent Effects in Acid-Catalyzed Biomass Conversion Reactions. Angew. Chemie—Int. Ed. 2014, 53 (44), 11872-11875.
(5) Lide, D. R. Handbook of Chemistry and Physics, 84th ed.; CRC Press, 2003.
6) Marianou, A. A.; Michailof, C. M.; Pineda, A.; Iliopoulou, E. F.; Triantafyllidis, K. S.; Lappas, A. A. Effect of Lewis and Brønsted Acidity on Glucose Conversion to 5-HMF and Lactic Acid in Aqueous and Organic Media. Appl. Catal. A Gen. 2018, 555 (2010), 75-87.
(7) Zhao, X.; Zhou, Z.; Luo, H.; Zhang, Y.; Liu, W.; Miao, G.; Zhu, L.; Kong, L. Z.; Li, S.; Sun, Y. γ-Valerolactone-Introduced Controlled-Isomerization of Glucose for Lactic Acid Production over Sn-Beta Catalyst. Green Chem. 2021.
(8) Walker, T. W.; Chew, A. K.; Li, H.; Demir, B.; Zhang, C. Z.; Huber, G. W.; Van Lehn, R. C.; Dumesic, J. A. Universal Kinetic Solvent Effects in Acid-Catalyzed Reactions of Biomass-Derived Oxygenates. Energy Environ. Sci. 2018, 0-24.
(9) Chew, A. K.; Walker, T. W.; Shen, Z.; Demir, B.; Witteman, L.; Euclide, J.; Huber, G. W.; Dumesic, J. A.; Van Lehn, R. C. Effect of Mixed-Solvent Environments on the Selectivity of Acid-Catalyzed Dehydration Reactions. ACS Catal. 2020, 10 (3), 1679-1691.
(10) Wong, C. Y. Y.; Choi, A. W. T.; Lui, M. Y.; Fridrich, B.; Horváth, A. K.; Mika, L. T.; Horváth, I. T. Stability of Gamma-Valerolactone under Neutral, Acidic, and Basic Conditions. Struct. Chem. 2017, 28 (2), 423-429.
(11) Jin, F.; Zhou, Z.; Moriya, T.; Kishida, H.; Higashijima, H.; Enomoto, H. Controlling Hydrothermal Reaction Pathways to Improve Acetic Acid Production from Carbohydrate Biomass. Environ. Sci. Technol. 2005, 39 (6), 1893-1902. https://doi.org/10.1021/es048867a.
(12) Prat, D.; Hayler, J.; Wells, A. A Survey of Solvent Selection Guides. Green Chem. 2014, 16 (10), 4546-4551.
(13) Prat, D.; Pardigon, O.; Flemming, H.-W.; Letestu, S.; Ducandas, V.; Isnard, O.; Guntrum, E. G.; Thomas, S.; Stéphane, R.; Cruciani, P.; Hosek, P. Sanofi's Solvent Selection Guide: A Step Toward More Sustainable Processes. Org. Process Res. Dev. 2013.
(14) Casas, A.; Ramos, M. J.; Rodriguez, J. F.; Perez, A. Tin Compounds as Lewis Acid Catalysts for Esterification and Transesterification of Acid Vegetable Oils. Fuel Process. Technol. 2013, 106, 321-325.
(15) Harris, J. W.; Cordon, M. J.; Di Iorio, J. R.; Vega-Vila, J. C.; Ribeiro, F. H.; Gounder, R. Titration and Quantification of Open and Closed Lewis Acid Sites in Sn-Beta Zeolites That Catalyze Glucose Isomerization. J. Catal. 2016, 335, 141-154.

1. A method for producing a difructose anhydride comprising contacting a solution comprising fructose and gamma valerolactone (GVL) with an acidic catalyst at an effective temperature for an effective contact time to produce the difructose anhydride.

2. The method of claim 1, wherein the acidic catalyst is an acidic heterogenous catalyst.

3. The method of claim 2, wherein the acidic heterogenous catalyst comprises Brønsted acidic sites.

4. The method of claim 2, wherein the acidic heterogenous catalyst comprises Lewis acidic sites.

5. The method of claim 2, wherein the acidic heterogenous catalyst comprises a zeolite.

6. The method of claim 5, wherein the zeolite comprises Brønsted acidic sites.

7. The method of claim 6, wherein the zeolite comprises an Al-beta zeolite.

8. The method of claim 5, wherein the zeolite comprises Lewis acidic sites.

9. The method of claim 8, wherein the zeolite comprises a Sn-beta zeolite.

10. The method of claim 2, wherein the acidic heterogenous catalyst is a mesoporous material.

11. The method of claim 10, wherein the acidic heterogeneous catalyst comprises Brønsted acidic sites.

12. The method of claim 10, wherein the acidic heterogenous catalyst comprises Lewis acidic sites.

13. The method of claim 10, wherein the mesoporous material comprises SBA-15.

14. The method of claim 13, wherein the SBA-15 is functionalized with propylsulfonic acid.

15. The method of claim 1, wherein the acidic catalyst is a homogenous acidic catalyst.

16. The method of claim 1, wherein the concentration of fructose in GVL is between 0.0.5 wt % and 0.5.0 wt %.

17. The method of claim 1, wherein the effective contact time is less than 4 hours.

18. The method of claim 1, wherein the effective temperature is a temperature between 90° C. and 165° C.

19. A composition for producing a difructose anhydride comprising a solution, the solution comprising fructose and gamma valerolactone, in contact with the acidic catalyst according to claim 1.

20. A reactor system for producing a difructose anhydride comprising a reaction vessel having the composition according to claim 19 therein.
